SEQUENCE LISTING (6 sequences)

SEQ ID NO: 1 - DNA, 3386 bp, Homo sapiens
gtttttagttagtatgaacgggaaggcgagggtaccatttacagcgacgggtaaccacgcgggaggagcaggtcaactcccgacagtggaggtgaagatgcgtgcgcctcgcgcaccttgtccctcgcgcgacgccgtccgcgccgcccgcggttcggtcgggagcggcagcccgtgctccggttccggttccggttccggctggcgtctgcacctgcgaccaccgtgagcagtcatggcgtactccacagtgcagagagtcgctctggcttctgggcttgtcctggctctgtcgctgctgctgcccaaggccttcctgtcccgcgggaagcggcaggagccgccgccgacacctgaaggaaaattgggccgatttccacctatgatgcatcatcaccaggcaccctcagatggccagactcctggggctcgtttccagaggtctcaccttgccgaggcatttgcaaaggccaaaggatcaggtggaggtgctggaggaggaggtagtggaagaggtctgatggggcagattattccaatctacggttttgggatttttttatatatactgtacattctatttaagctatcaaaggggaaaacaactgcagaggatgggaaatgctatactgccatgcctggaaacacccacaggaaaattaccagttttgagcttgctcaactgcaagaaaaactgaaggagacagaagcagccatggaaaaattaatcaacagagtgggacctaatggtgagaggataagggcttctgattttccatgacttaggaaacagaattaagactgtttcattcattgaagatcagaatgccctgcgtacagtctggaaaagtaacatgtgccgcctgaagagacaagagtttgtgataagcagcagagcacagactgtgacttctgaccaagagaaacggttgctacatcagctccgagaaatcaccagggtcatgaaagaaggaaaattcattgacagattttctccagagaaagaagctgaggaggccccttacatggaggactgggaaggttaccctgaagagacttacccaatttatgacctttcagactgtatcaagcgtaggcaagaaacaatcttggtggattaccctgacccaaaagaactttctgctgaagaaatagctgaaagaatgggaatgatagaagaggaagaatcagatcatttgggttgggaaagtctgcccactgaccccagagcccaggaagataattctgttacctcgtgtgatccaaagccagaaacatgttcctgctgttttcatgaagacgaggatcctgctgtcttggcagagaatgctggattcagtgcagatagctaccctgagcaagaggaaaccaccaaagaagagtggtcccaagactttaaagatgaagggttgggcatcagcaccgataaagcatatacaggcagcatgctgaggaagcgtaacccccagggtttagagtgaaaacagccagtctgaagtatccattactcaagtcccaagatgggaccactcaggggcaaaggtctgactcttcctggtaggtgtaacagatagttcacctgtgaacgaacatcagcttacagatgatgaggacttaaggttgcaagaatgaagatttcagactccaagatcccttattctttgggccttgagcaggccctagctcttggttggtttatatataaggaatatatttgtgtatttatatatcagacatttggatgtacctttcagttatttaggatttagaaaattcaatatcctgtatgcgctatttcccaccagaggcagaggccacaggagataggccatggttcagccctgttctcttcctaaggccaggcccacaggggatactgaggaaccaccagccccttcaggcggcacagcagaccctgaagagaactgctctaacttaagtaccttctgacttcaccatttttccagcctctgggtgtggttctgatgaaccttaaacttgaaaggcacaggccagaggaaggacagtgttgacccacatgtcattggtgaggttatgatctctttgctattatcacctacttacccacctgaggctcctaggaaacaatgaatgaaaggtatagaaaaaaattaggaagtaaacagggcttttttcccctttatgaaaaaagtacacagaagtgttgaaacgacggcgccgccctggccccctgctgtagctgccgccttggttctcttggctgaagctggtgctgttgggcgcgtggctcccagctgcacactggcgagtgagtcggcctcttgaactgctcaggaaacatccccagatggagagagagaaacaggcagtgggtgagaccagagctcccacctgaacacagtccccagcacagcatttttgactgcaccccgagtcccaccagggcccctgtaccacttctacagcccacaggatcaggcccactcatctctactaggcaaagattccagggcaggcccagctggaaggaggcggcacccccagcccagtgtaatcaagtcaacagccagaccaaaacctgtgatcagccaacaaagtgcgcactcaaaagttctgggccaccaaaccatactgagcctgaacaggggcagggagaatgtctgagaggatatggccattgaagacggggaagaaagaacaaaggctgcatcttctggaagagaccacaaccagggcaagcactccagggagcctacgagggggctctgcggggatgagcccatgggaggctggggtgtaggagtgctcagaacaacaactctgggacacaagaagctacaggcaccaacagtgaggtctcagaagggtcccagctggtggcctccaaattctccatactgcagaaccccaatctgctcaaaatccttgaatggtttctgattgctcttaagctgaagaccaacattctctcccttcccctaggtctaaaataaacacatactccccacctctggggcaccaagagttgcagccacaccaggctggtctgttgcctcacatgcactgcactccctcttgccacagggcccttgctcctgctgcctgcactgccctccccctcgcccccctcagtgatcaactcccactcatcctgcagatttcagcttggagtcacctcccgagggacgctgtgcctgcctccctcgtcagagcaggtgttgtcatctactcacagaatctggcttccttccacaaagcacccccgtgagcatctgtttaacattgtctctccacctagactgtaaggtccgtgaaagcagggaccacacccatttgtgctcacttgggacctacagcctctaatccagagcctgacacaaacctcaataaatatttaagaactg

SEQ ID NO: 2 - DNA, 1958 bp, Homo sapiens
  CDS (236)..(1345)
gtttttagttagtatgaacgggaaggcgagggtaccatttacagcgacgggtaaccacgcgggaggagcaggtcaactcccgacagtggaggtgaagatgcgtgcgcctcgcgcaccttgtccctcgcgcgacgccgtccgcgccgcccgcggttcggtcgggagcggcagcccgtgctccggttccggttccggttccggctggcgtctgcacctgcgaccaccgtgagcagtcatggcgtactccacagtgcagagagtcgctctggcttctgggcttgtcctggctctgtcgctgctgctgcccaaggccttcctgtcccgcgggaagcggcaggagccgccgccgacacctgaaggaaaattgggccgatttccacctatgatgcatcatcaccaggcaccctcagatggccagactcctggggctcgtttccagaggtctcaccttgccgaggcatttgcaaaggccaaaggatcaggtggaggtgctggaggaggaggtagtggaagaggtctgatggggcagattattccaatctacggttttgggatttttttatatatactgtacattctatttaagctatcaaaggggaaaacaactgcagaggatgggaaatgctatactgccatgcctggaaacacccacaggaaaattaccagttttgagcttgctcaactgcaagaaaaactgaaggagacagaagcagccatggaaaaattaatcaacagagtgggacctaatggtgagagcagagcacagactgtgacttctgaccaagagaaacggttgctacatcagctccgagaaatcaccagggtcatgaaagaaggaaaattcattgacagattttctccagagaaagaagctgaggaggccccttacatggaggactgggaaggttaccctgaagagacttacccaatttatgacctttcagactgtatcaagcgtaggcaagaaacaatcttggtggattaccctgacccaaaagaactttctgctgaagaaatagctgaaagaatgggaatgatagaagaggaagaatcagatcatttgggttgggaaagtctgcccactgaccccagagcccaggaagataattctgttacctcgtgtgatccaaagccagaaacatgttcctgctgttttcatgaagacgaggatcctgctgtcttggcagagaatgctggattcagtgcagatagctaccctgagcaagaggaaaccaccaaagaagagtggtcccaagactttaaagatgaagggttgggcatcagcaccgataaagcatatacaggcagcatgctgaggaagcgtaacccccagggtttagagtgaaaacagccagtctgaagtatccattactcaagtcccaagatgggaccactcaggggcaaaggtctgactcttcctggtaggtgtaacagatagttcacctgtgaacgaacatcagcttacagatgatgaggacttaaggttgcaagaatgaagatttcagactccaagatcccttattctttgggccttgagcagaatatcctgtatgcgctatttcccaccagaggcagaggccacaggagataggccatggttcagccctgttctcttcctaaggccaggcccacaggggatactgaggaaccaccagccccttcaggcggcacagcagaccctgaagagaactgctctaacttaagtaccttctgacttcaccatttttccagcctctgggtgtggttctgatgaaccttaaacttgaaaggcacaggccagaggaaggacagtgttgacccacatgtcattggtgaggttatgatctctttgctattatcacctacttacccacctgaggctcctaggaaacaatgaatgaaaggtatagaaaaaaattaggaagtaaacagggcttttttcccctttatgaaaaaagtacacagaagtgttgaaacga

SEQ ID NO: 3 - protein, 369 aa, Homo sapiens
MAYSTVQRVALASGLVLALSLLLPKAFLSRGKRQEPPPTPEGKLGRFPPMMHHHQAPSDGQTPGARFQRSHLAEAFAKAKGSGGGAGGGGSGRGLMGQIIPIYGFGIFLYILYILFKLSKGKTTAEDGKCYTAMPGNTHRKITSFELAQLQEKLKETEAAMEKLINRVGPNGESRAQTVTSDQEKRLLHQLREITRVMKEGKFIDRFSPEKEAEEAPYMEDWEGYPEETYPIYDLSDCIKRRQETILVDYPDPKELSAEEIAERMGMIEEEESDHLGWESLPTDPRAQEDNSVTSCDPKPETCSCCFHEDEDPAVLAENAGFSADSYPEQEETTKEEWSQDFKDEGLGISTDKAYTGSMLRKRNPQGLE

SEQ ID NO: 4 - DNA, 2732 bp, Homo sapiens
gtttttagttagtatgaacgggaaggcgagggtaccatttacagcgacgggtaaccacgcgggaggagcaggtcaactcccgacagtggaggtgaagatgcgtgcgcctcgcgcaccttgtccctcgcgcgacgccgtccgcgccgcccgcggttcggtcgggagcggcagcccgtgctccggttccggttccggttccggctggcgtctgcacctgcgaccaccgtgagcagtcatggcgtactccacagtgcagagagtcgctctggcttctgggcttgtcctggctctgtcgctgctgctgcccaaggccttcctgtcccgcgggaagcggcaggagccgccgccgacacctgaaggaaaattgggccgatttccacctatgatgcatcatcaccaggcaccctcagatggccagactcctggggctcgtttccagaggtctcaccttgccgaggcatttgcaaaggccaaaggatcaggtggaggtgctggaggaggaggtagtggaagaggtctgatggggcagattattccaatctacggttttgggatttttttatatatactgtacattctatttaagctatcaaaggggaaaacaactgcagaggatgggaaatgctatactgccatgcctggaaacacccacaggaaaattaccagttttgagcttgctcaactgcaagaaaaactgaaggagacagaagcagccatggaaaaattaatcaacagagtgggacctaatggtgagagcagagcacagactgtgacttctgaccaagagaaacggttgctacatcagctccgagaaatcaccagggtcatgaaagaaggaaaattcattgacagattttctccagagaaagaagctgaggaggccccttacatggaggactgggaaggttaccctgaagagacttacccaatttatgacctttcagactgtatcaagcgtaggcaagaaacaatcttggtggattaccctgacccaaaagaactttctgctgaagaaatagctgaaagaatgggaatgatagaagaggaagaatcagatcatttgggttgggaaagtctgcccactgaccccagagcccaggaagataattctgttacctcgtgtgatccaaagccagaaacatgttcctgctgttttcatgaagacgaggatcctgctgtcttggcagagaatgctggattcagtgcagatagctaccctgagcaagaggaaaccaccaaagaagagtggtcccaagactttaaagatgaagggttgggcatcagcaccgataaagcatatacaggcagcatgctgaggaagcgtaacccccagggtttagagtgaaaacagccagtctgaagtatccattactcaagtcccaagatgggaccactcaggggcaaaggtctgactcttcctggtaggtgtaacagatagttcacctgtgaacgaacatcagcttacagatgatgaggacttaaggttgcaagaatgaagatttcagactccaagatcccttattctttgggccttgagcagcggcgccgccctggccccctgctgtagctgccgccttggttctcttggctgaagctggtgctgttgggcgcgtggctcccagctgcacactggcgagtgagtcggcctcttgaactgctcaggaaacatccccagatggagagagagaaacaggcagtgggtgagaccagagctcccacctgaacacagtccccagcacagcatttttgactgcaccccgagtcccaccagggcccctgtaccacttctacagcccacaggatcaggcccactcatctctactaggcaaagattccagggcaggcccagctggaaggaggcggcacccccagcccagtgtaatcaagtcaacagccagaccaaaacctgtgatcagccaacaaagtgcgcactcaaaagttctgggccaccaaaccatactgagcctgaacaggggcagggagaatgtctgagaggatatggccattgaagacggggaagaaagaacaaaggctgcatcttctggaagagaccacaaccagggcaagcactccagggagcctacgagggggctctgcggggatgagcccatgggaggctggggtgtaggagtgctcagaacaacaactctgggacacaagaagctacaggcaccaacagtgaggtctcagaagggtcccagctggtggcctccaaattctccatactgcagaaccccaatctgctcaaaatccttgaatggtttctgattgctcttaagctgaagaccaacattctctcccttcccctaggtctaaaataaacacatactccccacctctggggcaccaagagttgcagccacaccaggctggtctgttgcctcacatgcactgcactccctcttgccacagggcccttgctcctgctgcctgcactgccctccccctcgcccccctcagtgatcaactcccactcatcctgcagatttcagcttggagtcacctcccgagggacgctgtgcctgcctccctcgtcagagcaggtgttgtcatctactcacagaatctggcttccttccacaaagcacccccgtgagcatctgtttaacattgtctctccacctagactgtaaggtccgtgaaagcagggaccacacccatttgtgctcacttgggacctacagcctctaatccagagcctgacacaaacctcaataaatatttaagaactg

SEQ ID NO: 5 - DNA, 1530 bp, Homo sapiens
  CDS (765)..(1391)
caaaggaaggaggaactaacttgtggaatgctgagaaaggtaaaaacaccttcatataaggaagaggaacaggctatgacctcatgcttgctttgacaagtataagcatgccagggcaaatatttaggctaaactgtgggagctaagaacagttgatttctttattatggctagcagatatctaagcatgttagcacaggttcttgaataaattttgcttctaagagaagttactattcctaattatatggggagtaaagtctctttgaagaggaatctctactttactttttacacttgtgctttgataattttttacttggaaacctcatcttttggcgtttttttttttgagctatttctctagtgttgctatttgatttaatttctgaccttcatttttgtttcccaaccttttttttttgtttgatatgaggggttccgtgactgaggttctgagctgctgttgatgtgctatacctccttcaattctcagctatcaaaggggaaaacaactgcagaggatgggaaatgctatactgccatgcctggaaacacccacaggaaaattaccagttttgagcttgctcaactgcaagaaaaactgaaggagacagaagcagccatggaaaaattaatcaacagagtgggacctaatggtgagaggataagggcttctgattttccatgacttaggaaacagaattaagactgtttcattcattgaagatcagaatgccctgcgtacagtctggaaaagtaacatgtgccgcctgaagagacaagagtttgtgataagcagcagagcacagactgtgacttctgaccaagagaaacggttgctacatcagctccgagaaatcaccagggtcatgaaagaaggaaaattcattgacagattttctccagagaaagaagctgaggaggccccttacatggaggactgggaaggttaccctgaagagacttacccaatttatgacctttcagactgtatcaagcgtaggcaagaaacaatcttggtggattaccctgacccaaaagaactttctgctgaagaaatagctgaaagaatgggaatgatagaagaggaagaatcagatcatttgggttgggaaagtctgcccactgaccccagagcccaggaagataattctgttacctcgtgtgatccaaagccagaaacatgttcctgctgttttcatgaagacgaggatcctgctgtcttggcagagaatgctggattcagtgcagatagctaccctgagcaagaggaaaccaccaaagaagagtggtcccaagactttaaagatgaagggttgggcatcagcaccgataaagcatatacaggcagcatgctgaggaagcgtaacccccagggtttagagtgaaaacagccagtctgaagtatccattactcaagtcccaaggccctagctcttggttggtttatatataaggaatatatttgtgtatttatatatcagacatttggatgtacctttcagttatttaggatttagaaaattc

SEQ ID NO: 6 - protein, 208 aa, Homo sapiens
MCRLKRQEFVISSRAQTVTSDQEKRLLHQLREITRVMKEGKFIDRFSPEKEAEEAPYMEDWEGYPEETYPIYDLSDCIKRRQETILVDYPDPKELSAEEIAERMGMIEEEESDHLGWESLPTDPRAQEDNSVTSCDPKPETCSCCFHEDEDPAVLAENAGFSADSYPEQEETTKEEWSQDFKDEGLGISTDKAYTGSMLRKRNPQGLE

(19) United States
(12) Patent Application Publication
(10) Pub. No.: US 2004/0242513 A1
(43) Pub. Date: Dec. 2, 2004

(54) Tub antisense constructs

(76) Inventors: Dirk Prawitt, Mainz (DE); Bernhard Ulrich Zabel, Mainz (DE); Thomas Brueckmann, Mainz (DE); Erwin Schmidt, Mainz (DE); Andreas Winterpacht, Mainz (DE); Thomas Hankeln, Mainz (DE)

Correspondence Address: BROWDY AND NEIMARK, P.L.L.C., 624 NINTH STREET, NW, SUITE 300, WASHINGTON, DC, 20001-5303, US

(21) Appl. No.: 10/473,411

(22) PCT Filed: Mar. 28, 2002
(86) PCT No.: PCT/US02/09474

(51) Int. Cl. A61K 48/00; C07H 21/04; C07K 14/47; C07K 16/18

(52) U.S. Cl. 514/44; 536/23.5; 530/350; 530/388.25; 435/69.1; 435/320.1; 435/325

(57) ABSTRACT

Splice variants of a human “TUB” antisense gene have been discovered, which overlap with human TUB and murine “tub” in antisense orientation. The overlapping portions suggest a regulatory/attenuation effect, due to RNA-RNA-interaction. These findings demonstrate that these TUB antisense constructs can regulate weight disorders, such as obesity, and progressive sensorineural degeneration phenotypes of the central nervous system. Accordingly, the invention provides therapeutic methods utilizing these constructs, as well as methods for identifying compounds that can therapeutically treat weight disorders.

### CROSS-REFERENCE TO RELATED APPLICATIONS

This application claims the benefit of priority from U.S. provisional application No. 60/279,768, filed Mar. 30, 2001, the entire contents of which are incorporated herein entirely by reference.

### BACKGROUND OF THE INVENTION

### DESCRIPTION OF THE RELATED ART

Obesity represents the most prevalent of body weight disorders, and it is the most important nutritional disorder in the western world, with estimates of its prevalence ranging from 30% to 50% within the middle-aged population. Other body weight disorders, such as anorexia nervosa and bulimia nervosa, which affect approximately 0.2% of the female population of the western world, pose serious health threats.

Defined as “an excess of body fat relative to lean body mass,” obesity also contributes to other diseases. For example, this disorder is responsible for increased incidences of diseases such as coronary artery disease, hypertension, stroke diabetes, hyperlipidemia and some cancers (Nishina, et al., 1994; and Grundy, S. M. and Barnett, J. P., 1990). Obesity is not merely a behavioral problem; rather, it is the result from differences in both metabolism and neurologic/metabolic interactions. In part, these differences are the result of differences in gene expression, or levels of gene products or activity (Friedman, et al. 1991).

The epidemiology of obesity strongly shows that the disorder exhibits inherited characteristics (Stunkard, 1990). Moll et al. (1991), have reported that, in many populations, obesity seems to be controlled by a few genetic loci. In addition, human twin studies strongly suggest a substantial genetic basis in the control of body weight, with estimates of heritability of 80-90% (Simopoulos, A. P. and Childs B., 1989; and Borjeson, 1976).

Studies of non-obese persons who deliberately attempted to gain weight by systematically over-eating were found to be more resistant to weight gain, having the ability to maintain an elevated weight only by very high caloric intake. In contrast, spontaneously obese individuals with normal or moderately elevated caloric intake experience bodyweight gain. In addition, it is a commonplace experience in animal husbandry that different strains of swine, cattle, etc., have different predispositions to obesity. Studies of the genetics of human obesity and of models of animal obesity demonstrate that obesity results from complex defective regulation of both food intake, food induced energy expenditure and of the balance between lipid and lean body anabolism.

A number of models exist for the study of obesity (Bray, 1992; and Bray, 1989). For example, animals having mutations that lead to syndromes for which obesity is a symptom also have been identified. Attempts have been made to utilize such animals as models for the study of obesity, and the best studied animal models, to date, for genetic obesity are mice. For reviews, see e.g., Friedman, J. M. et al. (1991); and Friedman, J. M. and Liebel, R. L. (1992).

Studies utilizing mice have confirmed that obesity is a very complex trait with a high degree of heritability. Mutations at a number of loci have been identified which lead to obese phenotypes. These include the autosomal recessive mutations obese (“ob”), diabetes (“db”), fat (“fat”) and tubby (“tub”).

Noben-Trauth et al., (1996) identified a candidate gene for murine tub on distal chromosome 7. The tub gene in tubby-mutant mice differs in transcript size from that in normal mice (6.6 kb in tubby versus 6.3 kb in unaffected B6 mice). This difference is the result of a G-to-T transversion in the mutant gene, which abolishes one donor splice site in the 3′ coding region. This transversion leads to inclusion of parts of an intron in the tub-transcript, yielding the larger transcript. The Tub RNA transcript in tubby mice was also 4-fold more abundant than the transcript in normal mice. This mutation in the mouse causes maturity-onset obesity, insulin resistance, retinal degeneration, and neurosensory hearing loss (Coleman and Eicher 1990); and Heckenlively et al., 1995).

A number of researchers have investigated the function of the TUB gene and the protein it expresses. For instance, the human homolog of the tubby (“TUB”) gene resides in 11p15.3 and can be used as a linkage marker for the study of familial obesity in humans (Jones, et al., 1992). Human TUB shows 94% sequence identity to the mouse protein, with a particularly high conservation of the C-terminal half of the molecule. Expression analysis (i.e., Northern and RT-PCR) revealed that the candidate gene, “TUB,” is expressed primarily in the brain North et al., 1997). It also is known that the gene is expressed both spatially and temporally, which suggests a similarity in function of the TUB protein with carboxypeptidase E (“CPE”). The CPE gene is the site of a null mutation in the fat/fat mouse. The particular expression pattern of the TUB gene also suggests a similarity in function of TUB protein with prohormone convertase (“PCSK1”), which is involved in the pathogenesis of one form of gross obesity (Kleyn et al., 1996).

Alternate splicing can be observed in the expression of TUB, which can result in two gene products that differ by virtue of the presence or absence of exon 4, as evidenced by Kleyn et al. (1996). Boggon et al. (1999) have proposed that TUB and tubby-like proteins are bipartite transcription factors, having C-terminal DNA binding domains and N-terminal transcription modulation domains. In addition, tubby-like protein 1 (“TULP1”), another family member with 60-90% amino acid identity, maps to human 6p21.3. “TULP1”, which is comparable to TUB protein, is involved in progressive retinitis pigmentosa, one aspect of which also in the murine tubby phenotype.

U.S. Patents disclosing gene constructs associated with TUB do exist (see, e.g., U.S. Pat. Nos. 5,955,306; 6,121,017; and 6,204,372). However, none of these patents discloses mutations in the TUB gene that relate to obesity in humans. Given the severity, prevalence and potential heterogeneity of obesity disorders, there exists a great need for the identification of genes involved in causing obesity. There is also a need for the identification of constructs that are able to affect the expression of such genes.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

Accordingly, it is an object of the invention to provide isolated nucleic acid molecules that are able to affect the expression of genes involved in weight disorders.

It is another object of the invention to provide a method for identifying compounds useful in treating a weight disorder.

It is a further object of the invention to provide a therapeutic compound useful in treating a weight disorder, as well as methods for using the same.

These and other objects will become apparent to a skilled worker, upon understanding the disclosure set forth herein.

In one context, the invention discloses an isolated nucleic acid molecule comprising a nucleotide sequence selected from the group consisting of SEQ ID NO:2, SEQ ID NO:4 and SEQ ID NO:5 as well as conservative variants thereof. A conservative variant preferably hybridizes under high stringency conditions to a nucleic acid sequence complementary to the nucleotide sequence of SEQ ID NO:2, SEQ ID NO:4, or SEQ ID NO:5.

The invention also provides therapeutic and diagnostic methods, which utilize one or more sequences of the invention. For instance, a nucleic acid molecule of the invention can be used to assay the expression of TUB, comprising contacting a biological sample, preferably containing mRNA, with one or more of these nucleic acid molecules, which is labeled.

The invention further provides a method for identifying a compound that is useful in treating a disorder associated with a mutated TUB-AS, which involves contacting a test cell with a test compound and thereafter measuring the level or activity of mutated TUB-AS, wherein the elevated mutated TUB-AS level or activity, as compared to a control, is indicative of a compound useful in treating disorders associated with weight loss; and a mutated TUB-AS level or activity that is reduced, as compared to a control, is indicative of a compound useful in treating disorders associated with weight gain.

Further aspects of the present invention are directed to a polypeptide encoded by the nucleic acid molecule of the present invention, such as a polypeptide having amino acid sequence of SEQ ID NO:3 or SEQ ID NO:6, and a molecule which includes the antibody binding portion of an antibody specific for the polypeptide.

### BRIEF DESCRIPTION OF THE DRAWINGS

FIG. 1 shows the genomic organization of human TUB and TUB antisense on the genomic level. The alternative splice variant of the TUB gene, which results in two overlaps between the TUB and TUB antisense transcripts (exon 2 TUB-antisense with exon 14 of TUB and exon 12 of TUB-antisense with exon 4 of TUB), is also shown.

FIG. 2 shows the exon organization of TUB-AS gene the three alternatively spliced versions, designated cDNA A, B and C, of the human TUB-AS gene.

FIG. 3 shows the exon structure of a hypothetical TUB-AS cDNA with all detected exons, where the nucleotide positions refer to SEQ ID NO:1.

FIGS. 4A and 4B show the genomic structure (FIG. 4A) and the location of exon-intron splice donor and acceptor sequences (FIG. 4B).

FIGS. 5A-5C show the exon structure of: TUB-antisense variant A (FIG. 5A), which is missing exons 5 and 9 and possibly includes exon 12 (cDNA A shown in FIG. 2) and where the nucleotide positions refer to SEQ ID NO:2; TUB-antisense variant B (FIG. 5B), which is missing exons 5, 9, 10 and 11 (cDNA B shown in FIG. 2) and where the nucleotide positions refer to SEQ ID NO:4; and TUB-antisense variant C (FIG. 5C), which is missing exon 8 and possibly includes exons 1, 2, 10, 11, and 12 (cDNA C shown in FIG. 2) and where the nucleotide positions refer to SEQ ID NO:5.

FIGS. 6A and 6B show Northern analysis of transcripts from various murine tissues with TUB-AS exons 1-3 (FIG. 6A) and exon 7 (FIG. 6B) cDNA probes. An approximately 4 kb transcript (variant with all exons) was observed for all TUB-antisense probes used. Additional bands were detected when hybridization probes were generated that included the transcript sections that overlap with splice versions for the TUB gene itself. The approximately 7 kb large band could be the TUB transcript (new splice variant). The small approximately 2 kb band could then be an alternative splice variant of the TUB antisense gene (e.g., TUB antisense variant A).

### DETAILED DESCRIPTION OF THE INVENTION

Expression profiling analysis of both human and mouse brain tissue discloses overlapping expression of TUB (human) and TUB-AS (human), and tub (murine) and tub-as (murine), respectively. The overlap between antisense tub-as (murine) and tub likely signifies a regulatory-attenuation effect, due to RNA/RNA-interaction of the two corresponding transcripts. Mutated tub-transcript in tubby mice is 4-fold more abundant than the transcript in normal mice. This suggests the lack of a negative regulatory effect in tub-as. Accordingly, the obesity phenotype in the tubby mutant mice presumably is the result of an altered tub-as transcript. It is thus quite probable that in human obesity patients with quantitative trait loci to 11p15.3, the obese phenotype is caused by a less active TUB protein or a TUB-AS mutation which results in a less active TUB protein. A mutation in TUB-AS could also lead to progressive sensorineural degeneration phenotypes of parts of the central nervous system (Usher 1C, retinitis pigmentosa, progressive hearing loss, blindness)

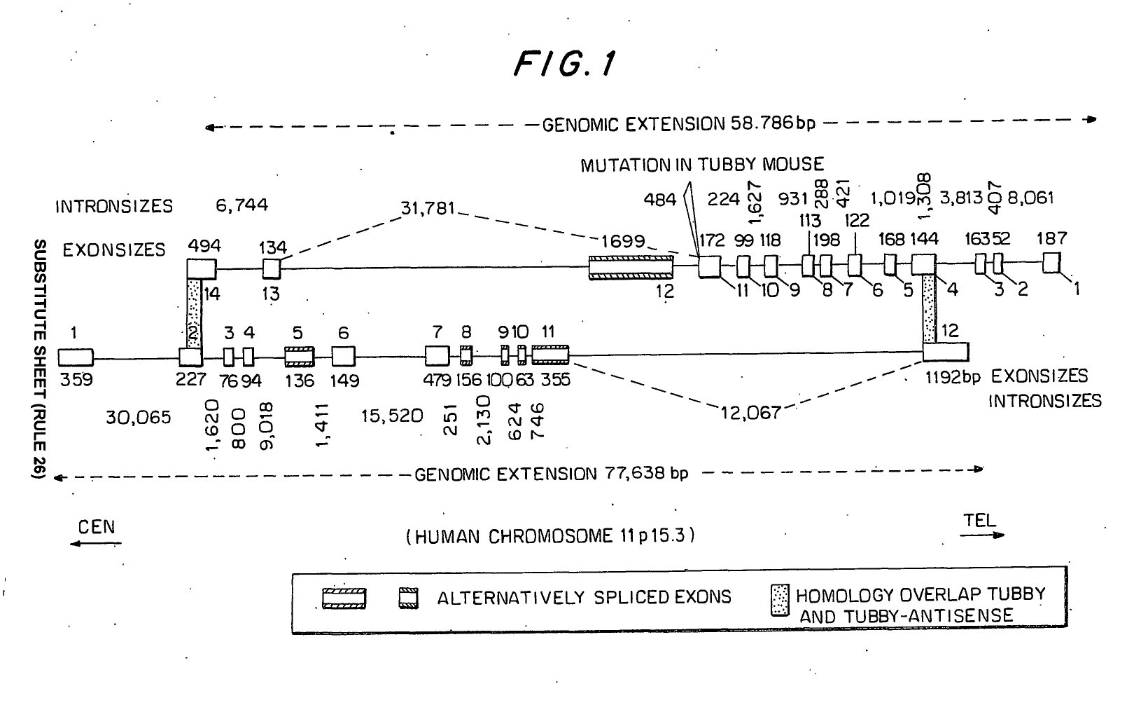

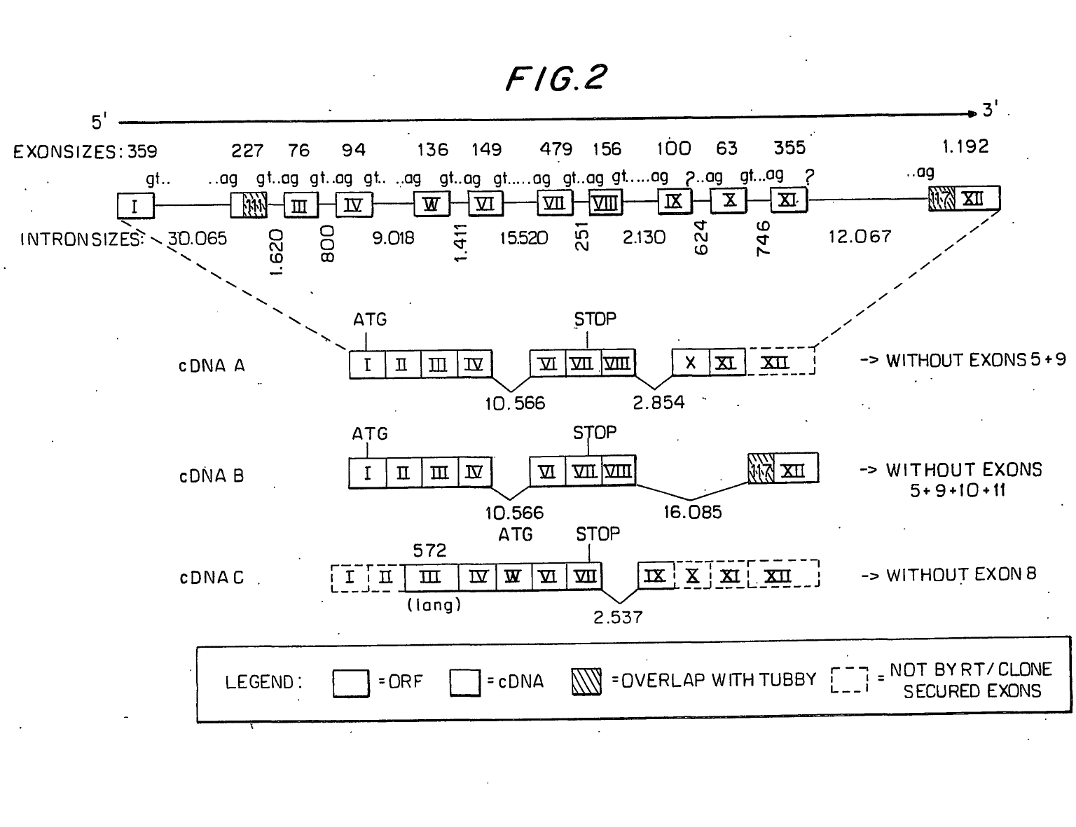

As part of the German (Deutschland) Human Genome Project (“DHGP”), the present inventors sequenced parts of the human chromosomal region 11p15.3 and the corresponding murine region. A comparative analysis between murine and human DNA revealed a TUB antisense (TUB-AS) that overlaps with both TUB and tub in antisense orientation (FIG. 1). Three alternatively spliced variants of this TUB-AS are shown in FIG. 2 and are denoted antisense (cDNA) variant A (SEQ ID NO: 2), antisense (cDNA) variant B (SEQ ID NO:4), and antisense (cDNA) variant C (SEQ ID NO:5). As shown in FIG. 1, antisense exons 2 and 12 overlaps respectively with exons 14 and 4 of TUB.

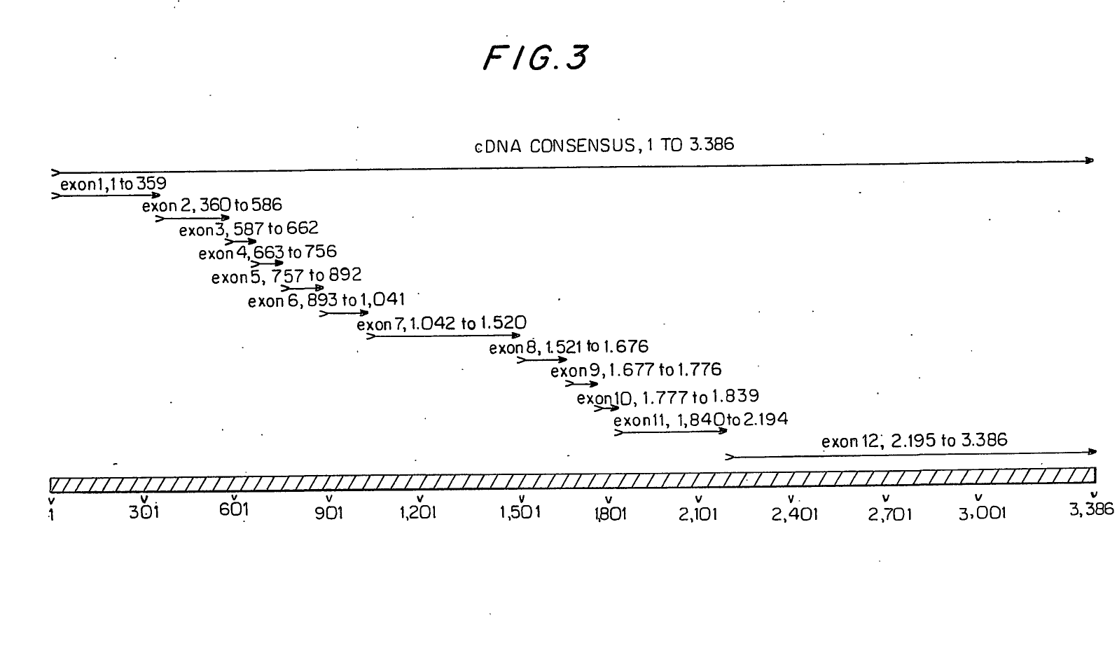

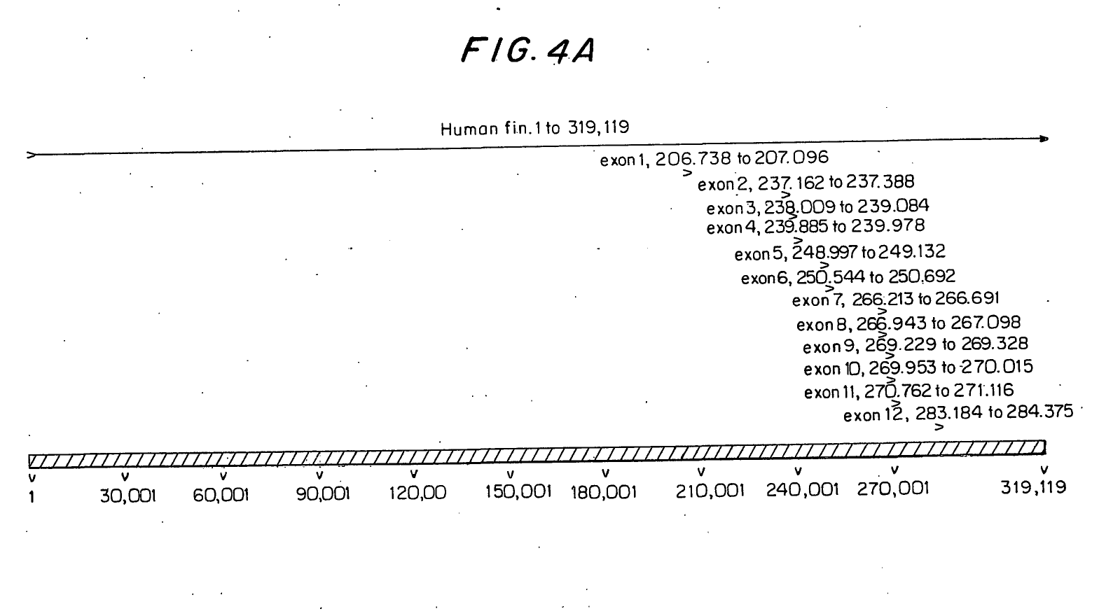

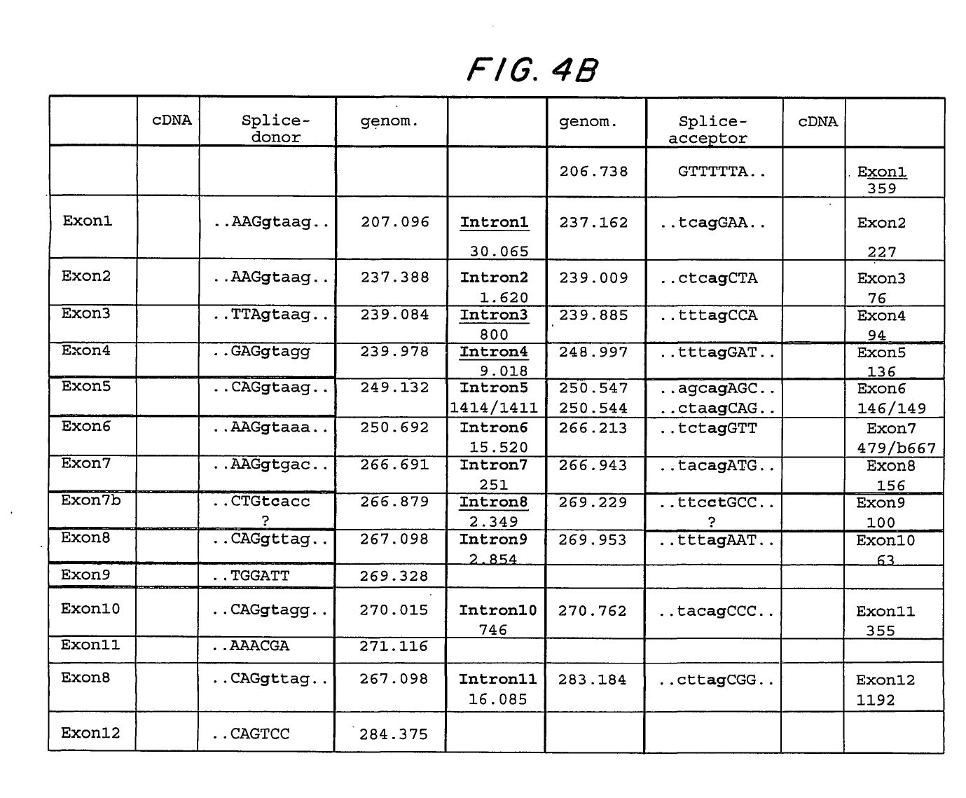

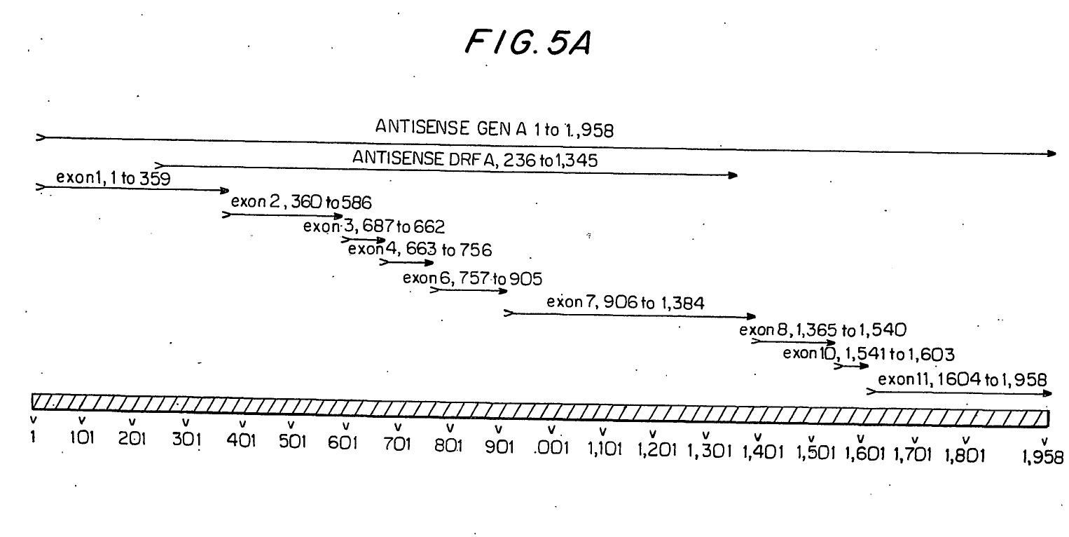

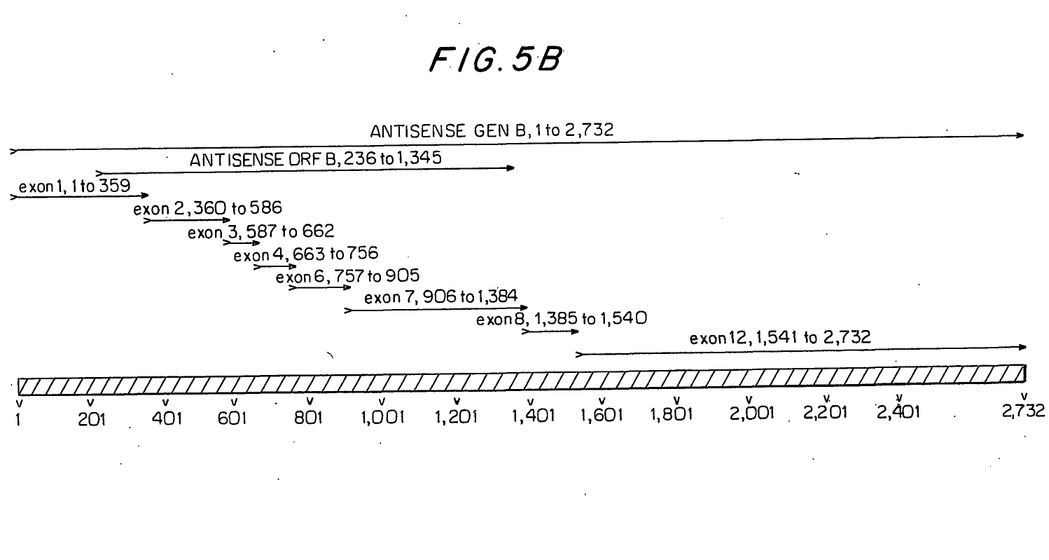

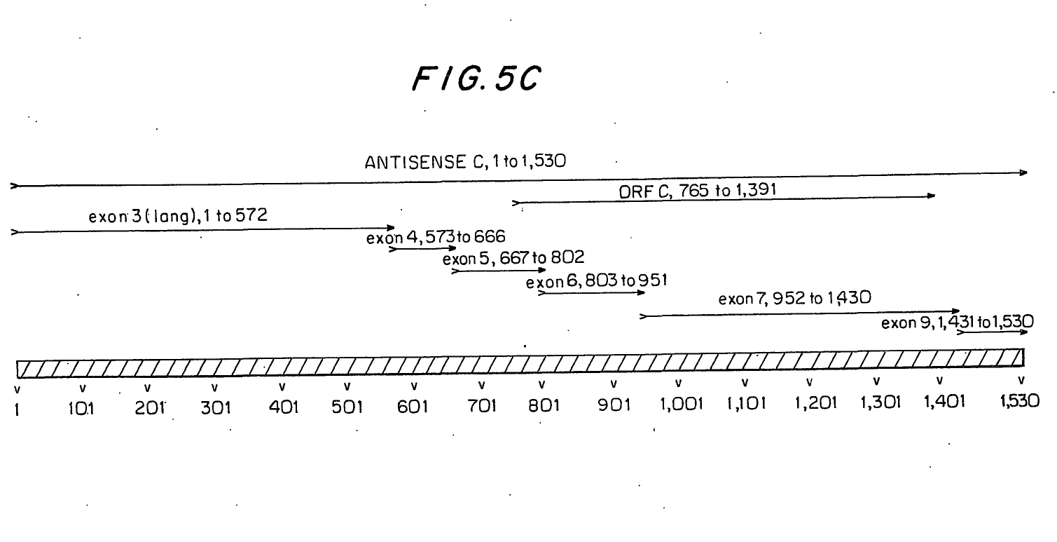

The exon structure of a hypothetical TUB-AS cDNA with all detected exons (SEQ ID NO:1) is presented in FIG. 3 and the genomic structure and location of exon-intron splice donor and acceptor sequences are presented in FIGS. 4A and 4B. In FIGS. 5A-5C, the exon structures of TUB antisense variant A (SEQ ID NO:2), antisense variant B (SEQ ID NO:4), and antisense variant C (SEQ ID NO:5) are shown. Overlap with the TUB gene was found at nucleotide positions 2195-2309 of SEQ ID NO:4. Possible polyadenylation signal sequences AATAAA were also found at nucleotide positions 2971-2976 and 3368-3373 of SEQ ID NO:1 and at nucleotide positions 2317-2322 and 2714-2719 of SEQ ID NO:4. Furthermore, ORFs in the three antisense variant sequence that can encode polypeptides were discovered. Antisense variants A and B have the same ORF which can encode a polypeptide having the sequence of SEQ ID NO:3, whereas antisense variant C has a different ORF which can encode a polypeptide having the sequence of SEQ ID NO:6. A good but not optimal Kozak sequence was also found surrounding the initiation codon in all three antisense variants.

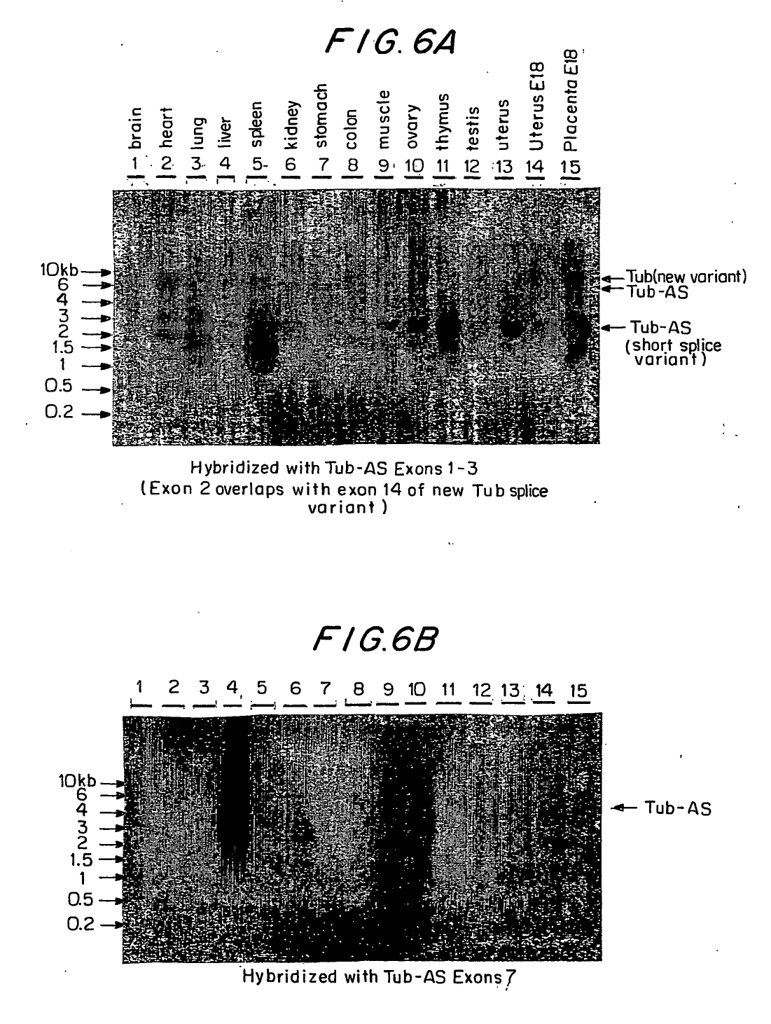

FIGS. 6A and 6B show Northern analysis of transcripts from various murine tissues with TUB-AS exons 1-3 (FIG. 6A) and exon 7 (FIG. 6B) cDNA probes. An approximately 4 kb transcript (variant with all exons) was observed for all TUB-antisense probes used. Additional bands were detected when hybridization probes were generated that included the transcript sections that overlap with splice versions for the TUB gene itself. The approximately 7 kb large band could be the TUB transcript (new splice variant). The small approximately 2 kb band could then be an alternative splice variant of the TUB antisense gene (e.g., TUB antisense variant A).

Further variants of the novel TUB-As sequences are those which hybridize under highly stringent conditions to the complement of SEQ ID NO:2, SEQ ID NO:4, or SEQ ID NO:5. Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al. (1984), as

Tm=81.5° C.+16.6 (log M)+0.41 (% GC)−0.61 (% form)−500/L

and for DNA:RNA hybrids, as

Tm=79.8° C.+18.5 (log M)+0.58 (% GC)−11.8 (% GC)²−0.56(% form)−820/L

where

M, molarity of monovalent cations, 0.01-0.4 M NaCl,

% GC, percentage of G and C nucleotides in DNA, 30%-75%,

% form, percentage formamide in hybridization solution, and

L, length hybrid in base pairs.

Tm is reduced by 0.5-1.5° C. (an average of 1° C. can be used for ease of calculation) for each 1% mismatching.

The Tm may also be determined experimentally. As increasing length of the hybrid (L) in the above equations increases the Tm and enhances stability, the full complement of SEQ ID NO:2, 4 or 5 can be used as the probe.

Filter hybridization is typically carried out at 68° C. (lower temperatures are used if formamide is added to compensate for the lowering of the hybridization temperature), and at high ionic strength (e.g., 5-6×SSC), which is non-stringent, and followed by one or more washes of increasing stringency, the last one being of the ultimately desired stringency. The equations for Tm can be used to estimate the appropriate Ti for the final wash, or the Tm of the perfect duplex can be determined experimentally and Ti then adjusted accordingly.

Hybridization conditions should be chosen so as to permit allelic variations and splice variants, but avoid hybridizing to other non-TUB antisense genes. In general, highly stringent conditions are considered to be a Ti of 5° C. below the Tm of a perfect duplex, and a 1% divergence corresponds to a 0.5-1.5° C. reduction in Tm. Typically, rat clones were 95-100% identical to database rat sequences, and the observed sequence divergence may be artifactual (sequencing error) or real (allelic variation). Hence, use of a Ti of 5-15° C. below, more preferably 5-10° C. below, the Tm of the double stranded form of the probe is recommended for probing a cDNA library.

As used herein, highly stringent conditions are those which are tolerant of up to about 15% sequence divergence. Without limitation, examples of highly stringent (5-15° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent wash conditions described above is hybridization in a solution of 6×SSC (or 6×SSPE), 5×Denhardt's reagent, 0.5% SDS, 100 μg/ml denatured, fragmented salmon sperm DNA at an appropriate incubation temperature Ti.

Preferred nucleic acid variants have a sequence with at least 75% sequence identity and more preferably 80% and even more preferably at least 85% sequence identity with a nucleotide sequence of SEQ ID NO:2, SEQ ID NO:4, or SEQ ID NO:5. Nucleic acids with at least 90%, more preferably 95%, and most preferably at least about 98-99% sequence identity with a nucleotide sequence of SEQ ID NO:2, SEQ ID NO:4, or SEQ ID NO:5 are particularly preferred embodiments of the present invention.

In addition to the methods set out below, the foregoing nucleic acids are useful in assaying for expression of TUB, for example by Northern Blot.

The present invention also provides for polypeptides encoded by the ORF in the nucleic acid molecule of the present invention. For instance, the nucleic acid molecule comprising the nucleotide sequence of SEQ ID NO:2 or SEQ ID NO:4 contains an ORF encoding for the same polypeptide of SEQ ID NO:3, whereas the nucleic acid molecule comprising the nucleotide sequence of SEQ ID NO:5 contains an ORF encoding for a different shorter polypeptide of SEQ ID NO:6. Accordingly, a polypeptide comprising the amino acid sequence of SEQ ID NO:3 or SEQ ID NO:6 is encompassed by the present invention. At least with regard to the polypeptide comprising the amino acid sequence of SEQ ID NO:6, a BLASTX search against a sequence database shows that the polypeptide has homology to an ATP-dependent serine protease, which may have utility in its ATP-binding and/or proteolytic activity.

Further provided by the present invention is a molecule which includes the antigen binding portion of an antibody specific for the polypeptide of the present invention and which is preferably an antibody.

It should be understood that when the term “antibodies” is used with respect to the antibody embodiments of the present invention, this is intended to include intact antibodies, such as polyclonal antibodies or monoclonal antibodies (mAbs), as well as proteolytic fragments thereof such as the Fab or F(ab′)2 fragments. Furthermore, the DNA encoding the variable region of the antibody can be inserted into other antibodies to produce chimeric antibodies (see, for example, U.S. Pat. No. 4,816,567) or into T-cell receptors to produce T-cells with the same broad specificity (see Eshhar, et al, 1990 and Gross et al, 1989). Single-chain antibodies can also be produced and used. Single-chain antibodies can be single-chain composite polypeptides having antigen binding capabilities and comprising a pair of amino acid sequences homologous or analogous to the variable regions of an immunoglobulin light and heavy chain (linked VH-VL or single-chain FV). Both VH and VL may copy natural monoclonal antibody sequences or one or both of the chains may comprise a CDR-FR construct of the type described in U.S. Pat. No. 5,091,513 (the entire content of which is hereby incorporated herein by reference). The separate polypeptides analogous to the variable regions of the light and heavy chains are held together by a polypeptide linker. Methods of production of such single-chain antibodies, particularly where the DNA encoding the polypeptide structures of the VH and VL chains are known, may be accomplished in accordance with the methods described, for example, in U.S. Pat. Nos. 4,946,778, 5,091,513 and 5,096,815, the entire contents of each of which are hereby incorporated herein by reference.

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

Polyclonal antibodies are heterogeneous populations of antibody molecules derived from the sera of animals immunized with an antigen.

Monoclonal antibodies (mAbs) are a substantially homogeneous population of antibodies to specific antigens. MAbs may be obtained by methods known to those skilled in the art. See, for example Kohler et al, (1975); U.S. Pat. No. 4,376,110; Harlow et al, (1988); and Colligan et al, (2001), the entire contents of which references are incorporated entirely herein by reference. Such antibodies may be of any immunoglobulin class including IgG, IgM, IgE, IgA, and any subclass thereof. The hybridoma producing the mAbs of this invention may be cultivated in vitro or in vivo. High titers of mAbs can be obtained by in vivo production where cells from the individual hybridomas are injected intraperitoneally into pristane-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Chimeric antibodies are primarily used to reduce immunogenicity during application and to increase yields in production, for example, where murine mAbs have higher yields from hybridomas but higher immunogenicity in humans, such that human/murine chimeric or humanized mAbs are used. Chimeric and humanized antibodies and methods for their production are well-known in the art, such as Cabilly et al (1984), Morrison et al (1984), Boulianne et al (1984), Cabilly et al, European Patent 0 125 023 (1984), Neuberger et al (1985), Taniguchi et al, European Patent 0 171 496 (1985), Morrison et al, European Patent 0 173 494 (1986), Neuberger et al, WO 8601533 (1986), Kudo et al, European Patent 0 184 187 (1986), Sahagan et al (1986); Robinson et al WO 9702671 (1987), Liu et al (1987), Sun et al (1987), Better et al (1988), and Harlow et al (1988). These references are hereby incorporated herein by reference.

A “molecule which includes the antigen-binding portion of an antibody,” is intended to include not only intact immunoglobulin molecules of any isotype and generated by any animal cell line or microorganism, or generated in vitro, such as by phage display technology for constructing recombinant antibodies, but also the antigen-binding reactive fraction thereof, including, but not limited to, the Fab fragment, the Fab′ fragment, the F(ab′)₂ fragment, the variable portion of the heavy and/or light chains thereof, and chimeric or single-chain antibodies incorporating such reactive fraction, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

Therapeutic Methods:

Gene regulation can take place at the transcriptional level, but can also be modified by mRNA editing or degradation. These processes involve proteins and small RNA molecules. Antisense transcripts might regulate gene expression by a mechanism such as: shared enhancers, promoter occlusion and post transcriptional gene silencing (PTGS). PTGS, a mechanism well know in plants and fungi, involves the formation of double-stranded RNA molecules. In human disease regions, such antisense transcripts also appear to play a significant role. For example, the gene UBE3A, which is responsible for the Angelman Syndrome, appears to be indirectly regulated by a paternally-expressed antisense transcript (Rougeulle et al., (1998)). Accordingly, inappropriate regulation (i.e., down regulation) of the antisense transcript could account for some of the unresolved cases of Angelman Syndrome.

A particular aspect of the present invention relates to the use of a nucleic acid molecule of the invention in a therapy, such as antisense therapy, that regulates the expression of a TUB (and/or TUB-AS) or TUB-related protein. Regulation of the expression of a TUB/TUB-AS or TUB-related protein can be an effective tool for treating a disorder associated with a mutated or abnormally upregulated TUB gene, for example. The treatable disorder can be, for example, obesity or a disorder characterized by progressive sensorineural degeneration of the central nervous system, e.g., Usher 1C, retinitis pigmentosa, progressive hearing loss, or blindness, and the method of treating such a disorder associated with the TUB or TUB-AS gene involves administering to a patient in need thereof a composition comprising the nucleic acid antisense molecule of the present invention or any contiguous at least 15 nucleotide portion thereof that exhibits TUB expression inhibiting activity.

The utility of the antisense transcript would be for post-transcriptional regulation of the TUB mRNA based on attenuation effects, RNAi effects (mRNA degradation), heteroduplex formation and/or other antisense-effects (modification of mRNA like methylation). Post-transcriptional regulation would thus downregulate the TUB transcript amount either on at the mRNA level, or also at the protein-level (as a secondary effect). The tubby mutation that is found only in mouse results in an aberrant C-terminal region of TUB, which means a less active TUB protein. According to this finding, together with the lack of functional mutations in the human obese patients coupled to chromosome 11p15, the present inventors believe that obese patients (with coupling to 11p15.3) would have a hyperactive TUB-AS, either due to increased transcript amount of TUB-AS (e.g., due to a promoter mutation) or gain-of-function mutations in the TUB-AS gene itself. The mutation in any case should not be in the shared sequence of TUB and TUB-AS, since this would have already been detected when patients were sequenced for TUB-mutations.

The functional model for TUB is in energy homeostasis. A TUB 3′-splice variant (lacking exon 12, but including new exons 13 and 14 that are absent in wild-type TUB) is shown in FIG. 1. The new TUB variant polypeptide encoded by this 3′-splice variant can either have a completely different function or more likely a modified function from wild-type TUB. For example, the wild-type TUB C-terminus is linked to the plasma membrane by PI(4,5)P2 (phosphoinositol biphosphate). Using a G-protein coupled protein interaction with PLC-beta, PI(4,5)P2 becomes PIP3 and TUB translocates into the nucleus, where it can bind to genes and regulate transcription (via C-terminal region). The TUB variant polypeptide of the present invention modifies the C-terminal structure. This could either result in a modified (or missing) linkage to the plasma membrane of the cell and/or a modified ability to regulate transcription of target genes in the nucleus. This means that it is possible that this cytoplasmic TUB readily (like the known TUB after phosphorylation) translocates into the nucleus, where it regulates specific gene transcription, without the need for phosphorylation. Accordingly, altered TUB-AS (either altered in dosage/level of transcript or is mutated/truncated) could post-translationally modify both the TUB wild-type and the TUB 3′-splice variants transcripts. In addition, an altered ratio of TUB wild-type to TUB 3′-splice variant would most likely have an effect on target gene transcription.

Tubby mice have a mutation that includes an intronic sequence in the transcript altering the C-terminal region of the protein (which is used to bind DNA, thus enabeling the transcription activity) and therefore disturbs the membrane binding/correct activation of target genes. Wild-type human TUB-AS should have a similar effect, by binding TUB post-transcriptionally, it can fine regulate/modify the transcript (the amount and perhaps the translationability and even methylation of sequence). The tubby mutation downregulates the available functional TUB, similar to an increase of TUB-AS levels (hyperactivity).

As used herein, “antisense” therapy refers to administration or in situ generation of nucleic acid molecules or their derivatives which specifically hybridize, e.g., bind, under cellular conditions, with the genomic DNA and/or cellular mRNA encoding one or more TUB or TUB-related gene products, so as to inhibit expression of that protein, for example, by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding of oligonucleotide sequences.

In a particular embodiment, the antisense construct is a nucleic acid which is generated ex vivo and that, when introduced into the cell, can inhibit gene expression by hybridizing with the mRNA and/or genomic sequences of a TUB gene. Such nucleic acids are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Alternatively, an antisense construct of the present invention can be delivered, for example, as part of an expression plasmid or vector that, when transcribed in the cell, produces RNA complementary to at least a unique portion of the cellular mRNA which encodes a TUB, TUB-AS or TUB-related protein. Additionally, general approaches to constructing oligomers useful in antisense therapy have been reviewed, for example, by Van der Krol et al. (1988) and Stein et al. (1988). With respect to antisense DNA, oligodeoxyribonucleotides derived from the translation initiation site, e.g., between the −10 and +10 regions of the TUB nucleotide sequence of interest, are preferred.

Antisense approaches can involve the design of oligonucleotides (either DNA or RNA) that are complementary to TUB mRNA and are based on the foregoing TUB-AS sequences. The antisense oligonucleotides will bind to the TUB mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In general, oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well (Wagner, R., 1994). Antisense oligonucleotides complementary to mRNA coding regions are less efficient inhibitors of translation but could be used in accordance with the invention. Whether designed to hybridize to the 5′, 3′ or coding region of TUB or TUB-AS mRNA, antisense nucleic acids should be at least six nucleotides in length, and are preferably less that about 100 and more preferably less than about 50 or 25 nucleotides in length. Typically they should be between 10 and 25 nucleotides in length. Such principals will inform the practitioner in selecting the appropriate oligonucleotides.

In a preferred embodiment, the antisense sequence is SEQ ID NO:2, SEQ ID NO:4, or SEQ ID NO:5. In addition, the antisense sequence can be an oligonucleotide sequence that consists of about 15-30, and more preferably 20-25, contiguous nucleotides of SEQ ID NO:2, SEQ ID NO:4, or SEQ ID NO:5.

Regardless of the choice of target sequence, it is preferred that in vitro studies are first performed to quantify the ability of the antisense oligonucleptide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989); Lemaitre et al., 1987); PCT Publication No. WO 88/09810, published Dec. 15, 1988) or the blood-brain barrier (see, e.g., PCT Publication No. WO 89/10134, published Apr. 25, 1988), hybridization-triggered cleavage agents. (See, e.g., Krol et al., 1988) or intercalating agents. (See, e.g, Zon, 1988). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxyethyl)uracil, 5-carboxymethylaminomethyl-2-thiouridine, 5-carboxymethylaminomethyluracil, dihydrouricil, beta-D-galactosylqueosine, inosine, N6-isopentenyladenine, 1-methylguanine, 1-methylinosine, 2,2-idimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987). The oligonucleotide is a 2′-0-methylribonucleotide (Inoue et al., 1987), or a chimeric RNA-DNA analogue (Inoue et al. (1987)).

Oligonucleotides of the invention may be synthesized by standard methods known in the art, e.g, by use of an automated DNA synthesizer (such as are commercially available from Biosearch, Applied Biosystems, etc.). As examples, phosphorothioate oligonucleotides may be synthesized by the method of Stein et al., 1988), methylphosphonate oligonucleotides can be prepared by use of controlled pore glass polymer supports (Sarin et al., 1988), etc.

The antisense molecules can be delivered to cells which express TUB in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous TUB transcripts and thereby prevent translation of the TUB mRNA.

For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981), the promoter contained in the 3′-long terminal repeat of Rous sarcoma virus (Yamamoto et al., 1980), the herpes thymidine kinase promoter (Wagner et al., 1981), the regulatory sequences of the metallothionein gene (Brinster et al., 1982), etc.

Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue; (e.g., for brain, herpesvirus vectors may be used), in which case administration may be accomplished by another route (e.g., systematically).

Endogenous TUB gene expression can also be reduced by inactivating or “knocking out” the TUB gene or its promoter using targeted homologous recombination (see, e.g, Smithies et al., 1985; Thomas and Capecchi, 1987; Thompson et al., 1989) each of which is incorporated by reference herein in its entirety). For example, a mutant, non-functional TUB (or a completely unrelated DNA sequence) flanked by DNA homologous to the endogenous TUB gene (either the coding regions or regulatory regions of the TUB gene) can be used, with or without a selectable marker and/or a negative selectable marker, to transfect cells that express TUB in vivo. Insertion of the DNA construct, via targeted homologous recombination, results in inactivation of the TUB gene. Such approaches are particularly suited in the agricultural field where modifications to ES (embryonic stem) cells can be used to generate animal offspring with an inactive TUB (e g., see Thomas and Capecchi, 1987; and Thompson, 1989). However this approach can be adapted for use in humans provided the recombinant DNA constructs are directly administered or targeted to the required site in vivo using appropriate viral vectors, e.g., herpes virus vectors for delivery to brain tissue; e.g., the hypothalamus and/or choroid plexus.

Alternatively, endogenous TUB gene expression can be reduced by targeting deoxyribonucleotide sequences complementary to the regulatory region of the TUB gene (i.e., the TUB promoter and/or enhancers) to form triple helical structures that prevent transcription of the TUB gene in target cells in the body (See generally, Helene, C., 1991; Helene et al., 1992; and Maher, L. J., 1992). Likewise, constructs of the present invention, by antagonizing the normal biological activity of one of the TUB proteins and mimicking the naturally occurring antisense regulation as described above, can be used in the manipulation of issue, e.g., lipid metabolism, both in vivo and for ex vivo tissue cultures.

Furthermore, like the antisense techniques (e.g., microinjection of antisense molecules, or transfection with plasmids whose transcripts are antisense with regard to a TUB mRNA or gene sequence), antagonizing the normal biological activity of one of the TUB proteins can be used to investigate the role of TUB in lipid metabolism. Such techniques can be utilized in cell culture, but can also be used in the creation of transgenic animals.

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Screening Assays

The invention also provides methods of screening or selecting compounds that are effective in treating disorders characterized by a mutation in the tub/TUB or TUB-AS gene, such as obesity. Accordingly, the invention provides a method for identifying compounds that are capable of treating a disorder associated with mutated forms of tub-AS or TUB-AS (e.g., obesity), which involves contacting a nucleic acid or oligonucleotide sequence of the invention in the presence and absence of a test compound with a cell, and determining whether the test compound upregulates or downregulates the levels of mutated forms of tub-AS or TUB-AS. In particular, the method provides for contacting a test cell with a test compound and thereafter measuring the level of mutated TUB-AS.

The test compound is a target pharmaceutical agent and may be of any make-up. The test cell can be any cell with an intact TUB coding/regulatory region, including TUB-AS. The test cell, for example, may be a cell transfected with the TUB coding/regulatory region. Levels of TUB-AS may be detected, for example, by Northern Blot. The mutated TUB-AS can be any mutation which affects the level of TUB-AS or the function of TUB-AS, i.e., promoter alteration, splice site mutation, alteration of functional important sequence, etc.

A compound that reduces mutated TUB-AS (i.e., level, activity) relative to a control can be used for treating disorders associated with weight gain, for example in treating obesity, since TUB-AS is capable of downregulating TUB. In the same vein, a compound that increases/introduces the TUB-AS levels (hyperactivity) relative to a control is useful in disorders associated with weight loss, i.e., positively affecting weight gain, for example in treating cachexia. Once a compound useful in affecting weight gain or loss is identified, it will be appreciated by those of skill in the art that such a known compound, i.e., pharmaceutical compounds, can be produced by routine experimentation and the production of such an identified compound is intended to be encompassed by the present invention. The present invention also includes compounds identified according to the foregoing method.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### References

Bemoist and Chambon, Nature 290:304-310 (1981)

Better et al, “Escherichia coli secretion of an active chimeric antibody fragment”, Science 240:1041-1043 (1988)

Boggon et al. Science 286(5447): 2119-25 (1999)

Boulianne et al, “Production of functional chimaeric mouse/human antibody”, Nature 312:643-646 (1984) Bray, Amer. J. Clin. Nutr. 5:891-902 (1989)

Bray, Prog. Brain Res. 93:333-341 (1992)

Brinster et al., Nature 296:39-42 (1982)

Cabilly et al, “Generation of antibody activity from immunoglobulin polypeptide chains produced in Escherichia coli.” Proc Natl Acad Sci USA 81:3273-3277 (1984)

Coleman and Eicher, J-Hered. 81(6):424-7(1990)

Colligan et al, Current Protocols in Immunology, Green Publishing Assoc., and Wiley Interscience, New York (2001)

Eshhar et al, “Chimeric T cell receptor which incorporates the anti-tumour specificity of a monoclonal antibody with the cytolytic activity of T cells: a model system for immunotherapeutical approach”, Br J Cancer Suppl, 10:27-29 (1990)

Friedman, et al., Mammalian Gene 1:130-144 (1991)

Friedman, J. M. and Liebel, R. L., Cell 69:217-220 (1992)

Friedman, J. M. et al., Mamm. Gen. 1:130-144 (1991)

Gautier et al., Nucl. Acids Res. 15:6625-6641 (1987)

Gross et al, “Expression of immunoglobulin-T-cell receptor chimeric molecules as functional receptors with antibody-type specificity”, Proc Natl Acad Sci USA, 86:10024-10028 (1989)

Grundy, S. M. and Barnett, J. P., Dis. Mon. 36:641-731 (1990)

Harlow et al, Antibodies: A Laboratory Manual, Cold Spring Harbor Laboratory, Cold Spring Harbor, New York (1988)

Heckenlively et al., Proc. Natl. Acad. Sci. U.S.A. 92(24): 11100-4. (1995)

Helene, Anticancer Drug Des. 6(6) :569-84 (1991)

Helene, et al. Ann. N.Y. Acad. Sci. 660:27-36 (1992)

Inoue et al., FEBS Lett. 215:327-330 (1987)

Inoue et al., Nucl. Acids Res. 15:6131-6148 (198.7)

Jones, et al. Genomics 14: 197-199 (1992)

Kleyn et al. Cell 85(2): 281-90 (1996)

Kohler et al, “Continuous cultures of fused cells secreting antibody of predefined specificity”, Nature 256:495-497 (1975)

Krol et al., BioTechniques 6:958-976 (1988)

Lemaitre et al., Proc. Natl. Acad. Sci. USA 84:648-652 (1987)

Letsinger et al., Proc. Natl. Acad. Sci. U.S.A. 86:6553-6556 (1989)

Liu et al, “Chimeric mouse-human IgG1 antibody that can mediate lysis of cancer cells”, Proc Natl Acad Sci USA 84(10):3439-3443 (1987)

Maher, L. J., Bioassays 14(12):807-15 (1992)

Moll et al., Am. J. Hum. Gen. 49:1243 (1991)

Meinkoth et al., “Hybridization of nucleic acids immobilized on solid supports”, Anal Biochem 138:267-284 (1984)

Morrison et al, “Chimeric human antibody molecules: mouse antigen-binding domains with human constant region domains”. Proc Natl Acad Sci USA 81:6851-6855 (1984)

Neuberger et al, “A hapten-specific chimaeric IgE antibody with human physiological effector function”, Nature 314:268-270 (1985)

Nishina, et al., Metab. 43:554-558 (1994)

Noben-Trauth et al., Nature 380:534-538 (1996)

North et al. PNAS 94:3128-3133 (1997)

Rougeulle et al., Nat-Genet. 19(1): 15-6 (1998)

S. Karger, Basel, Switzerland; Borjeson, M., Acta. Paediatr. Scand. 65:279-287 (1976)

Sarin et al., Proc. Natl. Acad. Sci. U.S.A. 85:7448-7451 (1988)

Sahagan et al, “A genetically engineered murine/human chimeric antibody retains specificity for human tumor-associated antigen” J Immunol 137:1066-1074 (1986)

Simopoulos, A. P. and Childs B., eds., in “Genetic Variation and Nutrition in Obesity”, World Review of Nutrition and Diabetes 63 (1989)

Smithies et al., Nature 317:230-234 (1985)

Stein et al., Cancer Res 48:2659-2668 (1988)

Stein et al., Nucl. Acids Res. 16:3209 (1988)

Stunkard, N. Eng. J. Med. 322:1483 (1990)

Sun et al, “Chimeric antibody with human constant regions and mouse variable regions directed against carcinoma-associated antigen 17-1A”, Proc Natl Acad Sci USA 84:214-218 (1987)

Thomas and Capecchi, Cell 51:503-512 (1987)

Thompson et al., Cell 5:313-321 (1989)

Van der Krol et al., Biotechniques 6:958-976 (1988)

Wagner et al., Proc. Natl. Acad. Sci. U.S.A. 78:1441-1445 (1981)

Wagner, R., Nature 372:333 (1994)

Yamamoto et al., Cell 22:787-797 (1980)

Zon, Pharm. Res. 5:539-549 (1988)

What is claimed is:

1. An isolated nucleic acid molecule, comprising a nucleotide sequence selected from the group consisting of SEQ ID NO:2, SEQ ID NO:4, and SEQ ID NO:5.

2. A polypeptide encoded by the nucleic acid molecule of claim 1.

3. The polypeptide of claim 2, comprising the amino acid sequence of SEQ ID NO:3.

4. A molecule which includes the antibody binding portion of an antibody specific for the polypeptide of claim 3.

5. The polypeptide of claim 2, comprising the amino acid sequence of SEQ ID NO:6.

6. A molecule which includes the antibody binding portion of an antibody specific for the polypeptide of claim 5.

7. A method of treating a disorder associated with the TUB or TUB-AS gene, comprising administering to a patient in need thereof a composition comprising the nucleic acid molecule of claim 1 or any contiguous at least 15 nucleotide portion thereof that exhibits TUB expression inhibiting activity.

8. The method of claim 7, wherein said any contiguous at least 15 nucleotide portion consists of 15-20 nucleotides.

9. A method of assaying expression of TUB, comprising:
contacting a biological sample containing mRNA with the nucleic acid molecule of claim 1, which is detectably labeled; and
detecting hybridization of said nucleic acid molecule with mRNA in said biological sample to assay for expression of TUB.

10. An isolated nucleic acid molecule that hybridizes under high stringency conditions to a nucleotide sequence complementary to the nucleotide sequence of claim 1.

11. An isolated antisense oligonucleotide molecule, comprising at least 15 contiguous nucleotides of said nucleotide sequence of claim 1.

12. The isolated antisense oligonucleotide molecule of claim 11, which consists of about 20-25 contiguous nucleotides.

13. A method of identifying a compound that is useful in treating a disorder associated with a mutated TUB-AS, comprising:
contacting a test cell with a test compound and thereafter measuring the level or activity of the mutated TUB-AS; and
identifying a compound that is useful for treating a disorder associated with a mutated TUB-AS, wherein a measured mutated TUB-AS level or activity that is elevated to a control is indicative of a compound useful in treating disorders associated with weight loss, and wherein a measured mutated TUB-AS level or activity that is reduced relative to a control is indicative of a compound useful in treating disorders associated with weight gain.

14. The method of claim 13, further comprising a step of producing said compound identified to be useful.

15. The method of claim 13, wherein the level of mutated TUB-AS is measured by Northern Blotting.

16. A compound identified by the method of claim 13 as being useful for treating a disorder associated with mutated TUB-AS.
